(19) United States
(12) Patent Application Publication
(10) Pub. No.: US 2006/0073203 A1
(43) Pub. Date: Apr. 6, 2006

(54) Dry polymer and lipid composition

(75) Inventors: Helena Ljusberg-Wahren, Hollviken (SE); Fredrik Joabsson, Lund (SE); Krister Thuresson, Bjarred (SE)

Correspondence Address: NIXON & VANDERHYE, PC, 901 NORTH GLEBE ROAD, 11TH FLOOR, ARLINGTON, VA, 22203, US

(73) Assignee: CAMURUS AB, Lund (SE)

(21) Appl. No.: 10/548,588

(22) PCT Filed: Mar. 12, 2004
(86) PCT No.: PCT/GB04/01099

§ 371 Date: Sep. 12, 2005

(30) Foreign Application Priority Data
Mar. 14, 2003 (GB) 0305941.7

(51) Int. Cl. A61K 31/57; A61K 31/716; A61K 31/717; A61K 31/728; A61K 9/26; A61K 31/715

(52) U.S. Cl. 424/469; 514/57; 514/60; 514/171; 514/55; 514/54

(57) ABSTRACT

The present invention provides an orally administrable composition comprising a dry mixture of polymer, lipid and bioactive agent, being capable on contact with water or GI tract liquid of forming particles comprising said lipid and said bioactive agent and optionally also water. It is preferable that such particles have a liquid crystalline phase structure. The invention also provides a method for the formation of compositions comprising polymer, lipid and bioactive agent.

This invention relates to orally administrable compositions containing bioactive agents, e.g. pharmaceutical, veterinary, or nutraceutical compositions, in particular compositions capable of controlled release of the bioactive agent.

For many orally delivered compositions containing bioactive agents, e.g. drugs, it is important that the agent be released from the other components of the composition in a controlled or sustained manner in order that the uptake of the agent from the gastrointestinal (GI) tract should occur over a predetermined (e.g. short or prolonged) period of time or in a particular region of the GI tract.

The most widely practised controlled release technique involves the use of compressed hydrophilic polymer matrices. Such matrices form a gel layer on hydration within the GI tract. This matrix can be erodible (e.g. soluble or biodegradable) or non-erodible, and porous or non-porous, and the bioactive agent is typically dissolved and/or dispersed in the matrix. Such conventional controlled release techniques are described for example in “Handbook of Pharmaceutical Controlled Release Technology”, Ed. Donald L. Wise, Marcell Dekker, New York, 2000.

Controlled release from non-erodible polymer matrices occurs via dissolution of the bioactive agent followed by its gradient-dependent diffusion through the gel layer, either through the swollen polymer network itself or through solvent-filled pores in the gel.

Where the bioactive agent is hydrophilic and highly soluble, it can be difficult to achieve sustained release as the bioactive agent is released relatively rapidly from the matrix. On the other hand, where the bioactive agent is hydrophobic or poorly water-soluble, it can be difficult to achieve a high degree of release of the agent from the matrix and moreover there is a risk that, once released, such agents may precipitate in the GI tract with the result that uptake from the GI tract may be unpredictable and highly variable.

In the alternative case of the erodible matrices, controlled release of the bioactive agent is achieved through erosion of the polymer matrix with the embedded bioactive agent being released from the eroding surface. The release rate is thus mainly determined by the rate of erosion of the matrix polymer. Highly soluble, hydrophilic bioactive agents may also be released by diffusion through the hydrated polymer matrix; however release by diffusion is often negligible for poorly water-soluble or hydrophobic bioactive agents. As with the non-erodible polymer matrices, there is also the problem of precipitation of such bioactive agents in the GI tract leading to unpredictable and highly variable uptake of the agent from the GI tract.

We have now found that these problems of the conventional controlled release techniques may be addressed by the use of hybrid matrices comprising a polymer and a lipid which, on contact with water, release self-assembled nanostructures, e.g. nanostructures having a liquid crystalline structure.

Thus viewed from one aspect the invention provides an orally administrable composition comprising a dry mixture of a physiologically tolerable hydrophilic polymer (preferably a gelable hydrophilic polymer), a physiologically tolerable lipid and a bioactive agent, said lipid, bioactive agent and polymer being interdispersed at a molecular level and being capable on contact with water of forming particles comprising said lipid and said bioactive agent and optionally also water.

The particles formed on contact with water are preferably emulsion droplets, micelles, particles of inverse micellar phase, vesicles, multilamellar bodies or aggregates or fragments of cubic, L3, lamellar or hexagonal phase liquid crystalline structures. With the lipid and polymer intermixed at the molecular level, such particles will assemble automatically on contact with water or GI tract liquids and will generally be nanometre-sized, e.g. with a maximum dimension on the nanometre to micrometer scale, e.g. 0.5 nm to 20 μm, more typically 10 to 5000 nm, especially 100 to 1000 nm.

Interdispersion of lipid and polymer at the molecular level cannot be achieved by techniques such as granulation, but particularly effectively be achieved by solvent removal from a solution of lipid and polymer in a common solvent or by mixing at elevated temperature and/or pressure, e.g. by melt extrusion.

Whether or not interdispersion at the molecular level has been achieved may readily be determined by scanning electronic microscopy of the composition; where a large proportion, e.g. >20% wt, of the lipid phase has collected as detectable droplets, e.g. of 500 nm or larger (more preferably of 100 nm or more), the admixing process will not have achieved the appropriate molecular level intermixing. Following admixture of the lipid and polymer at the molecular level, on storage some segregation may occur. The dispersion of the components will still however be superior to that achievable by granulation and the products are deemed still to be in accordance with the invention.

In an alternative approach, the composition may take the form of a polymer matrix containing pre-formed particles containing bioactive agent and lipid which on contact with water form (preferably liquid crystalline) nanoparticles, e.g. of L2, Lα, L3 cubic, or hexagonal phase.

Thus viewed from a further aspect the invention provides an orally administrable composition comprising a physiologically tolerable water soluble, hydrophilic polymer with dispersed therein particles comprising a physiologically tolerable lipid and a bioactive agent, which particles on contact with water or GI tract liquid form nanometre-sized particles (especially liquid crystalline particles) containing said lipid, said bioactive agent and water.

Again, by nanometre-sized is meant particles having a maximum dimension on the nanometre to micrometer scale, e.g. 0.5 nm to 20 μm, more typically 10 to 5000 nm, especially 100 to 1000 nm. In an alternative aspect, nanometre-sized as used herein may indicate particles on the nanomemter to micrometmer scale such as 10 nm to 100 μm, more typically 50 nm to 10 μm, especially 100 nm to 1 μm.

In one preferred embodiment, the compositions of the invention form small particles at low pH and larger particles at higher pH. Specifically, upon exposure to aqueous fluids at pH below 7, particularly below 3 and especially below 2.5, the particles formed may be 0.5 to 1000 nm particles, preferably 10 to 500 nm, most preferably 10 to 200 nm. In contrast, upon exposure to aqueous fluids at pH above 6.0, preferably above pH 7.0, particles of size 200 to 100 000 nm, preferably 250 to 10 000 nm and most preferably 400 to 5000 nm are formed (in some cases the particles will be >1000 nm).

The production of compositions containing such pre-formed liquid crystal precursor particles may be effected for example by dispersing the lipid and the polymer in a liquid in which the polymer is soluble but in which the lipid forms droplets, vesicles, particles of liquid crystalline phase etc, and then removing the solvent. The bioactive agent should be present dissolved or dispersed in the lipid.

In the case of such compositions, contact with aqueous fluids, e.g. the contents of the GI tract, causes the polymer matrix to release lipid particles containing water and the bioactive agent and having a liquid crystalline structure, for example L2, Lα, cubic, L3 or hexagonal phase, i.e. they are not simply structureless or water-unaffected droplets as in the case of a (simple) oil-in-water emulsion.

The compositions of the invention may be produced using appropriate combinations of components in order to achieve the desired phase behaviour in the end product. How to select the appropriate combinations is well within the normal capability of the skilled person but nonetheless it may be helpful here to review some simple rules in order to understand the phase behaviour of lipids, surfactants, and other amphiphilic compounds. Rather than specifying exact molecular structures or specific classes of substances it should be understood that the teaching applies for all compounds that are characterized by a bipolar structure with hydrophilic and hydrophobic moieties localised at separated positions. This provides this type of molecules with amphiphilic properties such that the hydrophilic parts have a preference for a polar environment while the hydrophobic parts have a preference for a non-polar environment. This is the reason such molecules assemble at interfaces between polar and non-polar regions and form molecularly organised phases.

The phase behaviour of all amphiphilic molecules is governed by the same type of physico-chemical rules. To be able to predict the phase behaviour of a given surfactant or lipid or, alternatively, to predict which compound to use to give the desired phase behaviour, some empirical rules have been shown to be useful (see Israelachvili, J. “Intermolecular and Surface Forces”, 2nd Edn., Academic Press, NY, 1991, and Jönsson et al. “Surfactants and Polymers in Aqueous Solution”, John Wiley & Sons, Chichester, 1998)

The “spectrum” of phase types can be considered to be substantially as set out below.

### CPP Value

| | | |
|---|---|---|
| Reversed micelles | >1 | Water-in-oil |
| Cubic | | |
| Reversed hexagonal | | |
| Cubic | | |
| Lamellar | 1 | Mirror plane |
| Cubic | | |
| Hexagonal | ⅓ to ½ | |
| Cubic | | |
| Micelles | <⅓ | Oil-in-water |

(where CPP, a dimensionless value, is v/1.a where v is the volume of the hydrophobic component of the amphiphile, 1 is the extended length of the hydrophobic component, and a is the maximum cross-sectional area of the amphiphile. In this scheme, the amphiphile can be considered to be conical in shape at either extreme with the hydrophilic group at the cone base in the micelles and at the cone point in the reversed micelles. On passing through the “mirror plane” between the extremes, the lamellar phase, the amphiphile can be considered to be cylindrical, i.e. its volume is simply its length times its maximum cross-sectional area so CPP=1).

The lamellar phase is often said to have a zero curvature, since the amphiphile film has no preference to curve in any direction. At the “oil-in-water” end of the scheme the structures curve towards oil giving “normal” aggregates, while at the “water-in-oil” end the structures curve towards water giving “reversed” aggregates.

A strong tendency to form films with a high curvature gives a preference for small spherical aggregates, like micelles, while a less pronounced tendency for curved films may give larger and more complicated aggregated structures. Thus, these are generally found for amphiphilic compounds that have a preference to give films with a curvature intermediate to that of micelles and the lamellar phase.

One way to characterise an amphiphilic compound is by the spontaneous curvature of the film. Its numerical value is calculated as the inverse of the radius of the curvature of the film. Essentially it can vary in between the inverse of the length of the amphiphile molecule to a similar negative value (with the lamellar phase at the mirror plane having a zero spontaneous curvature). While the spontaneous curvature is a useful concept to distinguish normal and reversed structures, it is not directly related to the molecular structure of the polar lipid or the amphiphilic compound in question.

A more useful way to be able to predict phase behaviour is to use the “critical packing parameter” (CPP) concept. CPP is calculated from geometrical considerations of the molecular structure of the amphiphilic compound as mentioned above. v and 1 set limits on how fluid chains pack together, on average, in an aggregate, and the mean molecular conformation thus depends on a, v, and 1. It is important to recognize that by a is meant an “effective” area. The relevance can be exemplified by the fact that the head group repulsion between ionic surfactants is strongly affected by screening electrolytes in a way such that it decreases with increasing electrolyte concentration. This means that the same ionic amphiphilic compound can, depending on the electrostatic screening situation, give different structures. Analogous situations are encountered with increasing temperature for non-ionic surfactants having an oligooxyethylene containing head group as well as for increasing concentration by themselves for many surface active compounds.

As mentioned above, a normal spherical micelle has a CPP-value below or equal to ⅓, the lamellar structure in the mirror plane has a CPP≈1, while the reversed structures are characterised by CPP-values higher than unity. The more complicated aggregated structures that typically are found in liquid crystalline phases (e.g. cubic and hexagonal) have intermediate CPP-values. For instance, a hexagonal structure has ⅓<CPP<½, while a bicontinuous cubic phase, which may have a saddle-shaped geometry with two principal radii of curvature with opposite sign, has a CPP-value close to unity.

As described above, surfactant geometry and packing determine the aggregate structure, and often it is found that single-chain surfactants form “normal” structures (e.g. micelles) while double-chain surfactants or lipids have a preference to form lamellar or reversed phases. It is also of utmost importance to recognise that a desired phase behaviour (and effective CPP-value) can be obtained by mixing two or more components of different CPP-value.

In this context it also deserves mention that another related empirical approach to characterise an amphiphilic molecule is Bancroft's rule:—a water-soluble emulsifier tends to give o/w emulsions, while an oil-soluble emulsifier tends to give w/o emulsions. This rule of thumb is mainly used in emulsion technology and has later been extended to the concept of hydrophilic-lipophilic balance (HLB). Based on the molecular structure, an amphiphilic compound can be assigned an HLB-number. The HLB-number can be calculated by summation of the HLB group numbers for the individual chemical groups that make up the amphiphilic compound. The HLB number of an amphiphilic molecule can then be used to predict whether normal or reversed emulsions are likely to form.

Thus HLB group number for certain hydrophilic and lipophilic groups are:

Hydrophilic Group Numbers

| | |
|---|---|
| —SO₄Na | 35.7 |
| —CO₂K | 21.1 |
| —CO₂Na | 19.1 |
| —N (tertiary amine) | 9.4 |
| Ester (sorbitan ring) | 6.3 |
| Ester (free) | 2.4 |
| —CO₂H | 2.1 |
| —OH (free) | 1.9 |
| -0- | 1.3 |
| —OH (sorbitan ring) | 0.5 |

Lipophilic Group Numbers

| | |
|---|---|
| —CF₃ | −0.870 |
| —CF₂⁻ | −0.870 |
| —CH₃ | −0.475 |
| —CH₂⁻ | −0.475 |
| —CH | −0.475 |

and HLB is calculated as 7 plus the sum of the hydrophilic and lipophilic group numbers. Where HLB is 3-6 the compound may find use as a w/o emulsifier, 7-9 as a wetting agent, 8-18 as an o/w emulsifier, 13-15 as a detergent, and 15-18 as a solubilizer.

For a system that gives “normal” structures, phase transformations and/or disintegration of the aggregated structures frequently take place on dilution with a large excess of an aqueous phase. This is in contrast to the phase behaviour of amphiphilic compounds that are characterised by reversed phases in equilibrium with excess water. Such behaviour can be found for amphiphilic molecules with CPP-values above unity. Both normal and reversed phases can be used in the present invention.

Compositions according to the invention will typically be produced as dry particulates: these can then be transformed into desired solid dosage forms, e.g. by compressing into tablets (optionally followed by coating, e.g. with a gastric acid resistant coating), filling into capsules, granulation, pelletization, grinding, etc. In such procedures, further components, e.g. tableting aids, binders, flavours, aromas, sweeteners, antioxidants, pH modifiers, viscosity modifiers, etc, may be added. All such dosage forms qualify as compositions according to the invention.

In the compositions according to the invention, which will generally be pharmaceutical or veterinary compositions or nutraceuticals, the bioactive agent may be hydrophilic, hydrophobic, amphiphilic or a substance which is solubilized within the GI tract, e.g. by virtue of the pH, intestinal flora, enzymes or cell surfaces encountered therein. Within the compositions themselves, the bioactive agent may be dissolved or dispersed within the lipid and/or within the polymer or interdispersed with either or both of these at the molecular level.

Preferably, the bioactive agent is dissolved or dispersed within the lipid or interdispersed therewith at the molecular level. This distribution of the bioactive agent may readily be achieved by dissolving it in the lipid (where it is lipid soluble) or by dispersing it within the lipid in particulate form, e.g. as a water-in-oil emulsion where it is water-soluble or as a fine powder (e.g. of nanoparticle size) where it is not lipid-soluble. Alternatively it may be dissolved or dispersed in a solvent in which the polymer and the lipid are soluble to form a solution or dispersion of lipid, polymer and bioactive agent from which the composition may be produced by solvent removal, e.g. by spray drying, lyophilization or evaporation, for example under reduced pressure.

Thus viewed from a further aspect the invention provides a process for the production of an orally administrable composition, preferably a composition according to the invention, which process comprises removing solvent from a solution of a physiologically tolerable hydrophilic gel-forming polymer, a physiologically tolerable lipid and a bioactive agent, and optionally grinding, compacting, coating and/or encapsulating the resultant solid. In this method it is preferable that the solvent (which may, obviously be a mixture of solvents) solubilises the polymer, lipid and bioactive agent as a molecularly mixed solution. Advantageously, the solvent should also be volatile to aid its removal from the mixture. Examples of suitable solvents include water, ethanol, isopropyl alcohol, formic acid, acetic acid (e.g. glacial or as a mixture with water), dichloromethane, chloroform, acetone, ethyl acetate and suitable mixtures thereof. Ethanol and acetic acid are particularly suitable.

Viewed from another aspect the invention provides a process for the production of an orally administrable composition, preferably a composition according to the invention, which process comprises melt extruding a mixture of a physiologically tolerable (generally hydrophilic and gel-forming) polymer, a physiologically tolerable lipid and a bioactive agent, and optionally grinding, compacting, coating and/or encapsulating the resultant solid.

Viewed from a still further aspect the invention provides a process for the production of an orally administrable composition, preferably a composition according to the invention, which process comprises removing solvent from a solution containing a dissolved physiologically tolerable water-soluble hydrophilic polymer and a dispersed physiologically tolerable lipid having dissolved or dispersed therein a bioactive agent.

Viewed from a yet still further aspect the invention provides a process for the production of an orally administrable composition, preferably a composition according to the invention, which process comprises removing solvent from a solution containing a dissolved physiologically tolerable water-soluble hydrophilic polymer, a dissolved or dispersed bioactive agent and a dispersed physiologically tolerable lipid. The lipid is preferably dispersed in the solution in the form of structured particles, especially nanometer particles of liquid crystalline phase (as described herein).

Besides lipid, polymer and bioactive agent, the solutions used in the processes of the invention may desirably also contain a surfactant with an HLB value in the range 8 to 18, e.g. a Tween, Cremophor, Solutol, Brij, Triton, etc. The surfactant may be ionic or nonionic, e.g. a sugar surfactant. Preferred sugar surfactants include sugar (especially sucrose) fatty acid esters, especially sucrose oleate, sucrose palmitate and/or sucrose laurate. Particular mention may also be made of surfactants with large polyoxyethylene head groups. Furthermore, to stabilise the (e.g. liquid crystalline) particulate dispersions or emulsions, the solutions used may desirably also contain an additional surface active polymer, e.g. a starch or starch derivative, a copolymer containing alkylene oxide residues (such as ethylene oxide/propylene oxide block copolymers), cellulose derivatives (e.g. hydroxypropylmethylcellulose, hydroxyethylcellulose, ethylhydroxyethylcellulose, carboxymethylcellulose, etc) or graft hydrophobically modified derivatives thereof, acacia gum, hydrophobically modified polyacrylic acids or polyacrylates, etc. The surface active polymer may also be used to provide a functional effect on the surface of the particles, for example, in order to selectively bind or target the particles to their desired site of action. In particular, polymers such as polyacrylic acids or chitosans may be used to provide mucus adhesive particles. Such particles will thus tend to remain localised at their site of release from the polymer matrix increasing the spacial control over the active agent release. Compositions of the invention comprising such surface modified particles form a further embodiment of the invention.

One combination of lipid and surfactant of particular note is the combination of a sugar surfactant (such as those indicated herein supra) and lipids or lipid mixtures comprising mono-, di- or tri-glycerides, particularly mono-glycerides and most preferably glyceryl monooleate. These combinations show highly desirable self-dispersing and self-emulsifying properties when used in the compositions or methods of the present invention.

The polymer used in the preparation of the compositions of the invention may be any polymeric material that serves to maintain the composition in solid form before contact with water and which serves to control the rate of release of lipid particles, e.g. liquid crystalline nanoparticles, from the composition after contact with water. A solid formulation (especially a free flowing powder, which is a preferred form) is advantageous not only for ease of dosing but also for ease of handling and processing during manufacturing.

Examples of suitable polymers include water-soluble and water-swellable polysaccharides (e.g. starch, starch derivatives such as maltodextrin, carrageenan, xanthan gum, locus bean gum, acacia gum, chitosan, alginates, hyaluronic acid, pectin, etc), cellulose derivatives—in particular cellulose ethers (e.g. methyl cellulose, hydroxyethyl cellulose, hydroxypropyl cellulose, hydroxypropyl methyl cellulose, hydroxyethyl ethyl cellulose, ethylhydroxyethylcellulose, carboxymethyl cellulose, etc), and synthetic polymers (e.g. polyacrylic acids, polyvinylpyrrolidone, polyalkylene oxides (for example polyethylene oxides or polyethylene glycols (PEGs)), etc.

The lipid used in the preparation of the compositions of the invention may be any swelling or non-swelling polar lipid, e.g. as defined by Small in the Journal of American Oil Chemists Society 45:108 (1968). Suitable examples of non-swelling polar lipids include: diglycerides, triglycerides, fatty acids (e.g. C₆₋₂₆ alkanoic and alkeneoic acids—the latter term including both singly and multiply ethylenically unsaturated acids), waxes, sterol esters, sterols (e.g. cholesterol, desmosterol, sitosterol, etc) C₆₋₂₆ alcohols, phytols, retinols, vitamins A, K, E and D, etc.

Suitable examples of swelling polar lipids include: galactolipids, lecithins, phosphatidylethanolamines, phosphatidylinositol, phosphatidylserine, sphingomyelin, monoglycerides, acidic soaps, cerebrosides, phosphatidic acid, plasmalogens, cardiolipins, di-, oligo-, poly-glycerolesters of fatty acids and -glycerolethers of fatty alcohols, etc.

The lipid, polymer and surfactant components of the compositions of the invention may each be single compounds; however it will generally be the case that two or more substances in one, two or three of these three categories be used. Some substances may fall into two or more such categories, e.g. polymers which are amphiphilic or which contain an amphiphilic component, such as for example acacia gum, may be used. These may play a dual or multiple role in the compositions, e.g. providing desired solidity, release profile, surface modification (e.g. for targeting or surface adhesion) and lipid phase stabilization. In the case of the lipids, it has surprisingly been found for example that the ability to load the released lipid particles with certain hydrophobic and amphiphilic bioactive agents is increased where the lipid comprises as at least one of its components a saturated or a mono or polyunsaturated C₆₋₂₆ fatty acid or a salt, ester or ether derivative thereof, e.g. oleic acid, linoleic acid, etc. or a salt, ester or ether thereof.

On contact of the molecular level mixed lipid/polymer compositions with water or gastrointestinal fluids, under the action of the water the polymer may form a gel while the lipid forms into particles containing lipid and bioactive agent. Where the polymer (e.g. polymer gel) is erodible, the lipid particles form and are released near the erosion boundary. Where the polymer (e.g. polymer gel) is porous or porosified by one of the components of the composition, lipid particles are formed within and diffuse out through the pores, in some cases fully formed, in other cases in forms which continue to take up water after release from the polymer matrix.

The lipids in the molecularly mixed lipid/polymer compositions may respond to water swelling of the polymer by forming structured or non-structured lipid particles, e.g. L₂, L_{α}, cubic or hexagonal phase liquid crystalline nanostructures, or L₃particles, micelles, microemulsion droplets or amorphous structures. Formation of the non-structured monoparticulates provides a particularly effective solubilizing vehicle for hydrophobic and amphiphilic bioactive agents; formation of L₂, L_{α}, cubic and hexagonal phase monostructures (which have separate hydrophilic, hydrophobic and amphiphilic microdomains) provides particularly effective solubilizing vehicles for hydrophilic, hydrophobic and amphiphilic bioactive agents as well as for mixtures of bioactive agents of different hydrophilicities/hydrophobicities.

Where the lipid is entrapped within the polymer in the form of pre-formed structured particles, these will consist of a liquid crystalline phase, e.g. a fragmented inverse micellar (L₂) phase, a fragmented lamellar (L_{α}) phase, a fragmented cubic phase, or a fragmented hexagonal phase. Such particles might contain lipid, water and bioactive agent—in the case of the L₂, L_{α}, L₃, cubic or hexagonal phased structures, the bioactive agent may be within a lipid or aqueous domain within the structures or, for an amphiphilic agent, at the boundary between such domains. However more generally the structured particles entrapped within the polymer may take the form of particles (e.g. solid, semisolid or fluid particles with a crystalline or amorphous structure) which takes up water to produce liquid crystalline nanostructures. The extent to which the particles maintain a structured form will depend at least in part upon the degree of drying carried out in order to render the composition “dry” as considered herein. Where the particles lose solvent to the extent that the original structure changes, exposure to the water in biological fluids will cause the particles to generate liquid crystalline nanostructures, for example with L₂, L_{α}, L₃, cubic or hexagonal phase structure.

Thus contact of water with the lipid/polymer compositions results in controlled (e.g. immediate, sustained and/or in use regiospecific) release into the water (in use into the GI tract) of lipid nanoparticles, generally 0.5 nm to 20 μm, more typically 10 to 5000 nm, especially 100 to 1000 nm in mode maximum dimension, containing the bioactive agent and from these, the bioactive agent is released (e.g. into the water such as the GI tract contents) at a rate that can be selected to optimize GI tract uptake and to minimize precipitation or over-rapid release and uptake. Alternatively, the rate of release can be controlled to give an immediate release if desirable.

The compositions according to the invention may thus be considered to comprise two essential sets of components: the precursors of the lipid nanostructures that are released from the composition on contact with water; and the precursors of the release rate determining matrix. Besides this, the compositions may of course, as mentioned above, comprise coating materials, binders, flavours, preservatives, etc.

The lipid nanostructure precursor comprises one or more bioactive agents, one or more lipids, optionally one or more surfactants, and optionally water. The nature of the lipid nanostructure released from the matrix is dependent on the physical mode of incorporation of the lipid (i.e. admixed with the polymer at the molecular level or embedded in the polymer as nanoparticles), as well as the chemical composition of the lipid/water/surfactant mixture. It is readily feasible to select the chemical composition and manner of incorporation of the precursor so as to cause the release of lipid nanoparticles of the desired nature, e.g. by performing phase behaviour studies of the reaction of the precursor components to water or other aqueous media using standard techniques, e.g. as discussed in “The Aqueous Phase Behaviour of Surfactants”, R. G. Laughlin, Academic Press, London, 1994.

The preparation of the lipid/polymer compositions of the invention can be achieved by at least two preferred processes as described above, i.e. by solvent removal from a solution of lipid and polymer and where present surfactant (with the bioactive agent dissolved or less preferably dispersed in the solution), or less preferably by solvent removal from a dispersion of lipid in a solution (generally but not essentially an aqueous solution) of the polymer. In this latter case, the bioactive agent will be dissolved or less preferably dispersed in the lipid phase which may also contain surfactant and/or water.

In an alternative method, the solvent removal from a dispersion of lipid in a solution (generally but not essentially an aqueous solution) of the polymer may be carried out with the bioactive agent dissolved in the polymer solution.

One advantage to the molecularly mixed state as opposed to the entrapped nanoparticle state is that it is less sensitive, e.g. to exposure to pressure during tableting or granulation. Furthermore, the composition resulting from the solvent removal may be the tablet precursor material, in which case this precursor material is formed in a single step.

Solvent removal may be effected by conventional techniques, e.g. solvent evaporation, lyophilization or spray drying, to give a “dry” material which can if necessary be powdered, granulated, tableted, coated, encapsulated, etc. to form solid dosage forms. By dry it is meant that the material may be compressed to form tablets. Preferably however a “dry” mixture, in powdered form, is stable and free flowing.

Where components of the compositions of the invention are heat sensitive, the solvent removal can be effected at ambient or sub-ambient temperatures, e.g. by lyophilization. Where active substances or excipients in the compositions are heat sensitive or labile, solvent removal will generally be preferred over processes involving elevated temperatures (e.g. melt extrusion) for the preparation of the compositions of the invention.

Compositions according to the invention may also be prepared by solvent removal (drying) from dispersions of lipid, polymer and bioactive agent in rigorously degassed water. Such degassing facilitates the mixing of the aqueous and non-aqueous phases and may reduce the need for emulsifiers or stabilizers.

Where the lipid/polymer mixture is produced from a dispersion of the lipid in a solution of the polymer, e.g. in water or an organic solvent, the lipid may first be admixed with bioactive agent and if desired surfactant and/or water before being dispersed using conventional techniques, e.g. high speed mixing, extrusion through a porous matrix, vortexing, sonication, high pressure homogenization, microfluidization, rotor stator mixing, etc. Dispersion will generally be into a solvent with the polymer then being added and solvent removal subsequently being effected. Where a surfactant is also added to the compositions, it is possible for them to form self dispersing mixtures in which case little if any mechanical dispersion will be necessary. Sugar surfactants as described herein are suitable in this method. If desired, e.g. to maintain a desired water content, the lipid may be added in solid or semi-solid particulate form (produced for example by cooling and pulverization or cold spraying), with the mixing with a solvent and the polymer or with a polymer solution and the subsequent solvent removal also being effected at sub-ambient temperatures. The energy input during dispersion may be selected so as to obtain the desired lipid particle size. If use of an organic solvent is to be avoided, if no common solvent can be found, or if incorporation of lipid nanoparticles with specific preferred structures is desired, then this pre-dispersion technique is to be preferred over the other technique involving solvent removal from a solution of lipid and polymer. Once again, where any of the components is heat sensitive, solvent removal is preferably effected at ambient or sub-ambient temperature, e.g. by lyophilization.

Where the bioactive ingredient has a tendency to crystallize, e.g. on solvent removal or on cooling to sub-ambient temperatures, the pre-dispersion technique may also be preferred as the bioactive agent is more likely to become trapped in a molecularly dissolved or solubilized form in the lipid particles.

In a particularly preferred embodiment, using the pre-dispersion technique, the bioactive agent may be dissolved in the lipid at a level such that in the resultant lipid/polymer mixture it is in a supersaturated state. Contact with fluids in the GI tract then results in release of lipid nanoparticles containing the bioactive agent in a metastable supersaturated state—such nanoparticles have been found to exert a higher potency in presenting the bioactive agent to the lining of the GI tract for absorption as compared to lipid particles in which the bioactive agent is in a normal stable state of dissolution. In a further preferred embodiment, the bioactive agent may be dissolved in the lipid at a level such that the resultant lipid/polymer mixture contains bioactive agent as a thermodynamically stable solution but generates lipid nanoparticles containing the bioactive agent in a metastable supersaturated state upon contact with fluids in the GI tract.

A further preferred technique for forming lipid/polymer compositions of the invention is melting and mixing (e.g. by melt extrusion or simply mixing at elevated temperature and optionally elevated pressure). This method is advantageous in that it is quick and simple to carry out, without requiring the removal of volumes of solvent. It is most suitable for use when the bioactive agent is not sensitive to elevated temperature, at least to the melting point of the mixture. Obviously, a method comprising a mixture of the “solvent removal” and “melt and mix” techniques may also be used, in which the ingredients including a relatively small amount of a suitable solvent (see supra) are mixed under somewhat elevated temperature and preferably also elevated pressure. A suitable mixture (e.g. a solution) may thus form with less solvent than would be required at ambient or sub ambient temperature but the temperature maintained lower than would be necessary for true melting of the components. The solvent may then be removed by reduction of pressure or by a later drying step using any suitable technique (e.g. the techniques indicated supra for the solvent removal method).

The compositions of the invention may also be formulated to contain materials which porosify the polymer matrix or which serve to produce gases on administration into the GI tract, e.g. compounds which are more water soluble than the polymer, fluorocarbons which are liquid below body temperature but gaseous at body temperature, or gas generators such as sodium hydrogen carbonate. These may facilitate lipid release or act to increase the buoyancy of the composition causing prolonged retention of the composition in the region of the GI tract where gas release occurs, for example the stomach.

Alternatively, retention of the composition in the stomach and/or control over the release profile may be achieved by trapping gas directly in the dry powder as part of the manufacturing process. This could occur, for example, as a result of including a dispersion of immiscible low boiling solvent (e.g. a fluorocarbon) in the mixture of solvent, polymer and active agent. When the bulk solvent is removed, the volatile solvent may evaporate producing pores, bubbles or other voids within the polymer. Similarly, gas could be generated by chemical means and trapped within the polymer matrix. After a porous matrix has formed, the initial gas may optionally be replaced by others if desired. This might be used, for example, to control the release profile of the active by altering how readily the gas dissolves in the aqueous fluid of the stomach. As the gas dissolves, the pores will more readily fill with fluid.

Once a dry powder of the lipid/polymer-hybrid has been obtained this can be further processed into solid dosage forms such as tablets, pellets, granules or capsules by conventional techniques, optionally using further excipients commonly employed in solid dosage forms such as fillers, binders, disintegration aids, glidants, lubricants, colours, flavours, sweeteners, taste-masking agents, and film-coating materials. However, due to the composition of the lipid/polymer-hybrid it is often not necessary to add binders or lubricants since the polymer or, respectively, lipid components of the lipid/polymer-hybrid are able to act as such during e.g. tableting. Due to this reason lipid/polymer-hybrid matrices are also particularly suitable for direct compression.

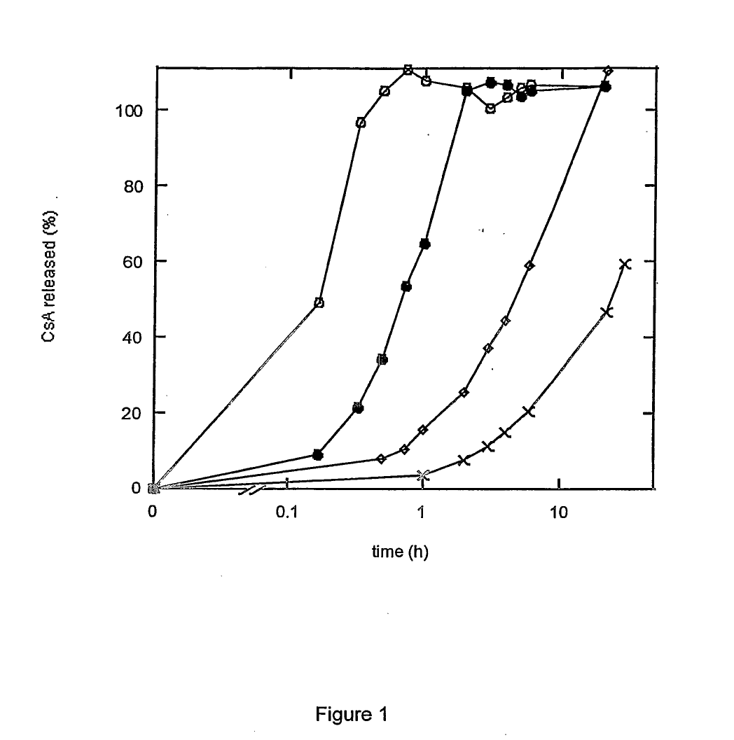

The release profile for the bioactive agent over time can be modified as desired by appropriate selection of the chemical nature and molecular weight of the polymer (as is illustrated in the Examples below). This is shown graphically in FIG. 1 of the accompanying drawings which shows the different release profiles for cyclosporin A (CsA) containing compositions in which the polymer is respectively (o) low molecular weight PVP, (•) high molecular weight PVP, (⋄) hydroxypropyl cellulose, and (x) hydroxypropylmethyl cellulose. The cyclosporin A is released from these compositions in the form of lipid carriers having CsA therein. PVP is used herein to indicate polyvinyl pyrrolidone.

A further advantage of the lipid/polymer-hybrids is that they are dry. By “dry”, as used herein, is indicated that they are functionally solid or semisolid, as opposed to fluid. Preferably, dry, as used herein indicates that the compositions may be broken, chopped, crushed or powdered, or otherwise formed into pieces of controlled size and/or shape. Such pieces may then be processed to enlarge, reduce or homogenise their granular sizes, coat them, mix them with binders or other agents and render them suitable for easy and handling in the manufacturing process (e.g as a uniform free flowing powder). The term “dry” may, but need not, imply the absence of solvents such as water and more generally indicates the function of a material having the properties of a dry or solid material. Thus, for example, a polymer matrix having trapped therein liquid crystalline lipid/water/active agent particles may function as a dry material in spite of the water content of the liquid crystalline particles.

The dry nature of the compositions of the present invention provides them with a considerable additional advantage. In particular, compositions containing lipid excipients are generally fluid, being, for example, in the form of an emulsion of active and lipid in water or in the form of an oily lipid formulation, such as an emulsion preconcentrate. Fluid formulations, however, present additional problems in terms of packaging and distribution, dosage and patient compliance when compared with dry formulations. Fluids are more difficult for patients to carry, measure and take than dry tablets and so a patient is less likely to comply correctly with their treatment regimen if they are given a fluid rather than tablets. Fluids may be packed into gelatin capsules but these are complex to manufacture and often large and unpleasant to swallow. The present dry formulations thus offer a considerable advantage in ease of administration while preserving the other advantages of lipid excipients. This advantage applies to “controlled” release formulations of all types, whether the control is in the form of immediate release (e.g. in the stomach) or whether the active agent is released in a gradual, delayed or selective manner, in one or more regions of the GI tract.

One challenge in formulation design is to find a composition that is suitable for the active compound in question. Active compounds can be sub-classed into three groups: hydrophilic substances characterised by a high aqueous solubility; hydrophobic (lipophilic) substances with low aqueous solubility but high solubility in oils; amphiphilic or membrane soluble substances that have a preference for interfaces between hydrophilic and hydrophobic domains (including membranes).

In order to better understand the advantages and possible uses of the present invention the three groups are briefly discussed.

Solubility enhancers are generally not needed for the first group and focus can be on obtaining a desired release profile. This can be accomplished with standard techniques. However, some hydrophilic substances with high aqueous solubility, e.g. peptides and proteins, which are administrated via the oral route, are sensitive to exposure to the hostile environment in the gastrointestinal tract (pH, enzymatic activity etc.) or may suffer chemical modification (e.g. ligand exchange). Others, e.g. heparin, have difficulties in permeating the intestinal mucosal membrane. The present invention can be used to overcome these problems. A substance sensitive to gastrointestinal tract exposure can be enclosed in hydrophilic domains within the lipid vehicles and in this way be protected from degradation. Lipid vehicles released from the solid matrices of the invention can also be designed to include lipids that mediate uptake. Thus for instance capric acid promotes absorption of large hydrophilic compounds, e.g. the peptide desmopressin. Furthermore, the released lipid particles can be surface modified by a suitable polymer (such as chitosan or derivatives thereof) so as to provide muco adhesive or other targeting properties. Another problem that can be encountered with certain active substances is a variable aqueous solubility, i.e. precipitating compounds (e.g. those which precipitate due to change in pH or on contact with the calcium in the GI tract). The lipid vehicles of the present invention can be used to either buffer a local environment inside the lipid vehicles or to retain a local milieu that promotes solubility of the active substances, also in ambient media in which the active substance has low solubility. Moreover, if transition to a state with low aqueous solubility occurs the drug molecules can be solubilised in other domains of the lipid vehicle.

The second group, comprising hydrophobic substances, generally needs solubility enhancers to be presented to the epithelial cells in sufficient quantities. This can be accomplished by using lipid vehicles of the invention that contain hydrophobic domains.

For the last class, amphiphilic or membrane soluble compounds, the invention offers unique possibilities, since nanostructure liquid crystalline phases are characterised by large interfacial regions. Thus, amphiphilic substances can be incorporated in high amounts resulting in high drug loads.

It should be noted that the advantages (protective properties and mediated uptake) mentioned in connection with the discussion about hydrophilic substances are also valid with the two latter classes of active substances. It should furthermore be recognised that substances sensitive to elevated temperatures, which may degrade during manufacturing using standard processes, e.g. melt extrusion, may conveniently be formulated and produced by taking advantage of the present invention.

Examples of bioactive agents that can be used in the compositions of the invention include but are not limited to progesterone, cyclosporin A, cyclosporin G, [O-(2-hydroxyethyl)-(D)Ser]⁸-cyclosporin, [3′-dehydroxy-3′-keto-MeBmt]¹-[Val]²-cyclosporin, bezafibrat, diltiazem, isradipin, verapamil, amphotericin B, coenzyme QlO, danazole, atovaquone, amlodipine, nifedipine, nimodipine, felodipine, paclitaxel, etoposide, irinotecan, tretinoin, sirolimus, tacrolimus, itraconazole, ketoconazole, propranolol, atenolol, atorvastatin, lovastatin, pravastatin, simvastatin, enalapril, lisinopril, candesartan, losartan, valsartan, olanzapine, sertraline, venlafaxine, mirtazepine, raloxifene, sildenafil, tadalafil, clarithromycin, azithromycin, ciprofloxacin, pioglitazone, rosiglitazone, atomoxetine, cilostazol, celecoxib, rofecoxib, diclofenac, ibuprofen, naproxen, aldosterone, betametasone, dexametasone, medroxyprogesterone, prednisolone, diazepam, flurazepam, lorazepam, midazolam, nitrazepam, and clomethiazol.

Especially conveniently the bioactive agent used in the present invention is one having a solubility in water at 20° C. of less than 1% w/v, more especially less than 0.01% w/v.

We have also found that cyclosporins and cyclosporin derivatives, in particular cyclosporin A, may particularly advantageously be formulated for administration dissolved in a fatty acid, e.g. a fatty acid containing up to 26 carbons, more particularly 6 to 20 carbons, for example an unsaturated fatty acid, especially a monounsaturated fatty acid, more especially a C₁₈ monounsaturated acid, more particularly oleic acid or also favourably linoleic acid.

We have further found that cyclosporins and cyclosporin derivatives, in particular cyclosporin A, may particularly advantageously be formulated for administration dissolved in a formulation comprising at least one fatty acid, e.g. a fatty acid containing up to 26 carbons, more particularly 6 to 20 carbons, for example an unsaturated fatty acid, especially a monounsaturated fatty acid, more especially a C₁₈ monounsaturated acid, more particularly oleic acid or also favourably linoleic acid. The formulations are preferably formulations according to the present invention.

When compounds such as cyclosporins, cyclosporin derivatives, cyclic peptides and in particular cyclosporin A are fomulated as a composition of the present invention with a fatty acid as indicated above then small (especially unimodal submicron) particles have been observed to form upon contact with an aqueous phase. These particles can contain a very high concentration of the bioactive agent and are thus an advantageous method of delivery of the bioactive since concentrated and supersaturated lipid compositions are thought to provide enhanced uptake. The formulation is also pH sensitive as indicated below.

In addition to giving an increased solubility to some sparingly soluble bioactive agents (such as cyclosporins, cyclosporin derivatives, cyclosporin A and certain cyclic peptides) fatty acids also have the advantage that they can change property depending on the surrounding pH. As a result, formulations comprising fatty acids change properties such as their phase behaviour, stability, solubility and such like depending upon the pH of the region of the GI tract. A fatty acid containing dry formulation, such as those described herein may thus, for example, release small (especially submicron) particles at low pH (stomach), while droplet size increases (e.g. to greater than half a micron, especially to greater than 1 micron) at higher pH (like in intestinal fluid). Destabilisation occurs at specific sites in GIT and this destabilisation may be related to phase change of the composition or released particles, precipitation and/or supersaturation of bioactive agent. As a result, the inclusion of a proportion of fatty acid in the lipid component of the formulations of the invention may provide further control over bioactive agent release and thus forms a further embodiment of the invention. The compositions of the invention which vary in particle size release with pH thus preferably contain a fatty acid.

In one embodiment of the invention, a composition of the invention contains a fatty acid and releases particles in auqeous solution at pH below 3 and larger particles in auqeous solution at pH above 6, wherein the particles released at pH below 3 are sub-nanometer in size. Preferably, the composition of the invention contains components including sufficient fatty acid to provide sub-micron (e.g. 0.5 to 1000 nm, preferably 1 to 250 nm, most preferably 10 to 150 nm) particles upon exposure to pH below 7, preferably below 3 and larger particles (e.g. 250 to 20 000 nm, preferably 400 to 5 000 nm) at pH above 6.0, preferably above 7. Such compositions may readily be prepared and identified by known methods (such as laser light scattering) and by reference to the Examples herein, especially Example 4 below.

We have also found that progesterone may particularly advantageously be formulated for administration dissolved in a C₆₋₁₀ alkanoic acid, particularly caprylic acid or in compositions comprising such fatty acids.

Thus viewed from a further aspect the invention provides a pharmaceutical composition comprising progesterone or a derivative thereof dissolved in a C₆₋₁₀ alkanoic acid or a physiologically tolerable salt thereof, said composition optionally and preferably containing a further physiologically tolerable lipid.

Such compositions may be formulated in any convenient dosage form, e.g. capsules, solutions, powders, tablets, etc., and conventional pharmaceutical carriers and excipients may be used.

The compositions however are preferably formulated as orally administrable compositions according to the earlier described aspects of the invention.

The invention will now be described further with reference to the following non-limiting Examples:

### EXAMPLE 1

Release of Droplets without Internal Structure Containing Progesterone from Formulations Solidified with Polyvinyl Pyrrolidone (PVP)

Two compositions were prepared using low and high molecular weight PVP (Plasdone K29/32 and K90 from ISP Technologies, Inc) and were then lyophilized. The composition contents for the two compositions are set out in Tables 1 and 2.

TABLE 1

| | Composition before | Composition after |
|---|---|---|
| Substance | lyophilization (%) | lyophilization (%) |
| Progesterone | 0.53 | 1.5 |
| Glycerol dioleate (GDO) | 2.1 | 5.9 |
| Caprylic acid | 2.1 | 5.9 |
| Cremophor RH (CrRH) | 4.2 | 11.8 |
| Polyvinyl pyrrolidone (PVP) | 26.7 | 75.0 |
| K29/32 | | |
| Ethanol (EtOH) | 64.4 | — |

TABLE 2

| | Composition before | Composition after |
|---|---|---|
| Substance | lyophilization (%) | lyophilization (%) |
| Progesterone | 0.19 | 1.5 |
| Glycerol dioleate (GDO) | 0.75 | 5.9 |
| Caprylic acid | 0.75 | 5.9 |
| Cremophor RH (CrRH) | 1.5 | 11.8 |
| Polyvinyl pyrrolidone (PVP) | 9.6 | 75.0 |
| K90 | | |
| Ethanol (EtOH) | 87.2 | — |

Since ethanol solubilises the components in the formulation, a molecular mixture was obtained. The formulation was solidified by removing ethanol with lyophilization. This gave a solid formulation which could be compressed to tablets (approximately 200 mg) by compression in a KBr IR-tablet press.

The release profile was then studied using a simulated intestinal fluid (SIF), pH=7.4, with the composition set out in Table 3.

TABLE 3

| Substance | Amount | |
|---|---|---|
| KH₂PO₄ | 34 | g |
| NaOH | 7.8 | g |
| Deionized water | 500 | ml |

The tablets were placed in baskets (rotating at 100 rpm) in 500 ml SIF at 37° C. in an Erweka dissolution bath. Upon contact with this excess aqueous phase, microemulsion droplets without internal structure were released from the solid formulation which carry the active substance. At each time where a data point was obtained, aliquots were withdrawn (100 μl) and analyzed with HPLC to obtain the progesterone concentration in the dissolution media at that time. The droplets had sizes below 1 μm. The release profiles seemed to be first order; and the release rate could be controlled by changing the molecular weight of the solidifying polymer. (Increasing the PVP molecular weight reduced the release rate)

### EXAMPLE 2

Release of Droplets without Internal Structure Containing Cyclosporin A from Formulations Solidified with Polyvinyl Pyrrolidone (PVP)

As in Example 1, two formulations were prepared, lyophilized and compressed to tablet form using Plasdone K29/32 and K90. The composition contents are set out in Tables 4 and 5 below respectively.

TABLE 4

| | Composition before | Composition after |
|---|---|---|
| Substance | lyophilization (%) | lyophilization (%) |
| Cyclosporin A (CsA) | 5.0 | 10.7 |
| Maizine-35 | 5.8 | 12.5 |
| Cremophor RH-40 | 7.1 | 15.3 |
| Oleic acid | 2.1 | 4.4 |
| Propylene glycol | 1.6 | 3.5 |
| Polyvinyl | 24.8 | 53.6 |
| pyrrolidone (PVP) | | |
| (Plasdone K29/32) | | |
| Ethanol (EtOH) | 53.7 | — |

TABLE 5

| | Composition before | Composition after |
|---|---|---|
| Substance | lyophilization (%) | lyophilization (%) |
| CsA | 1.8 | 10.7 |
| Maizine-35 | 2.1 | 12.5 |
| Cremophor RH-40 | 2.6 | 15.3 |
| Oleic acid | 0.75 | 4.4 |
| Propylene glycol | 0.59 | 3.5 |
| Polyvinyl pyrrolidone | 9.1 | 53.6 |
| (PVP) (Plasdone K90) | | |
| Ethanol (EtOH) | 83.0 | — |

Upon contact with an aqueous phase in excess using simulated intestinal fluid (SIF) (as in Example 1) microemulsion droplets without internal structure were released from the solid formulation which carry the active substance. The droplets mainly had sizes below 1 μm. The release profiles were first order and the rate could be controlled by changing the molecular weight of the solidifying polymer. Again increasing molecular weight retarded release.

### EXAMPLE 3

Release of Droplets without Internal Structure Containing Cyclosporin A from Formulations Solidified with Cellulose Based Polymers

This Example shows that if a solvent cannot be found that gives a molecular mixture of all the components that should be included in the formulation, it may be feasible to produce a polymer/lipid hybrid from an aqueous solution in which lipid aggregates form. This route is expected to give a tablet in which the lipid aggregates to some extent retain their structure from the aqueous phase.

The composition of the formulation without the solidifying polymer is given in Table 6. A high concentration of cyclosporin A is enabled by using oleic acid in the formulation. 10% of the formulation is emulgated in water and mixed with an aqueous solution of a cellulose based polymer (1% (HPC) or 2% (HPMC)). The mixture was lyophilized to obtain solid lipid formulations that contained a 1:1 weight ratio polymer:formulation (see Table 7).

Tablets were prepared by compression in KBr IR-tablet press. Upon contact with an aqueous phase (simulated intestinal fluid (SIF)) in excess, drug containing droplets without internal structure formed. The droplets mainly had sizes below 1 μm. Drug release profiles were obtained by using baskets (rotating at 100 rpm) in 500 ml SIF at 37° C. in a Erweka dissolution bath. At each time where a data point was obtained, aliquots were withdrawn (100 μl) and analyzed with HPLC to obtain the CsA concentration in the dissolution media at that time.

As in the earlier Examples the release kinetics could be controlled by changing the solidifying polymer.

TABLE 6

| Substance | Amount (wt %) |
|---|---|
| CsA | 20.0 |
| EtOH | 13.3 |
| Maizine-35 | 23.4 |
| Cremophor RH-40 | 28.5 |
| Oleic acid | 8.3 |
| Propylene glycol | 6.5 |

TABLE 7

| Substance | Amount (wt %) |
|---|---|
| CsA | 11.5 |
| Maizine-35 | 13.5 |
| Cremophor RH-40 | 16.4 |
| Oleic acid | 4.8 |
| Propylene glycol | 3.7 |
| HPC or HMPC | 50.0 |

### EXAMPLE 4

Selection of Composition for Drying

The following example illustrates a procedure for selection of drug composition that displays a phase change upon dilution in a media where fatty acids dissociate but exhibits stability in acid media where no such dissociation occurs. This formulation can be dried for instance by the route outlined in Example 3.

A Cyclosporine containing liquid formulation (see Table 8a) was manufactured in the following way. Cyclosporin A and ethanol were weighed into a glass vial and closed with rubber stopper and aluminium cap. The vial was placed on a rotating table until the substance was dissolved into the ethanol. The rest of the excipients were then added to the Cyclosorin A solution and the vial was again closed and placed on the rotating table for at least 2 hours. The resulting liquid composition was inspected in polarized light in order to determine that the sample was homogenous and free of crystals before use.

TABLE 8a

| Substances | Content % w/w |
|---|---|
| Cyclosporin A | 20 |
| Ethanol | 9 |
| Propylenglycol | 9 |
| Oleic acid | 19 |
| Cremophore RH 40 | 44 |

The phase change of the samples was monitored as an increase in particle size with time after dispersion in aqueous media. The experiments were performed in the following way. Drops of the self dispersing formulation were added directly on the surface of the degassed aqueous medium in the sample compartment of the particle sizer (Coulter LS230) and this dispersion procedure was continued until the PIDS obscuration value exceeded 45%. The media used to disperse the formulations were simulated gastric fluid (SGF) and simulated intestine fluid (SIF). SGF was prepared by adding 2 g sodium chloride and 7 mL hydrochloric acid into 1000 mL water, (pH approximately 1.2) and SIF was prepared by adding 6.8 g potassium phosphate and 190 mL 0.2M sodium hydroxide to 400 mL water, followed by adjustment of pH to 7.5±0.1 before the finally dilute with water to 1000 mL.

TABLE 8b

| | Dispersion | Dispersion |
|---|---|---|
| | with pH 1.5 | with pH 7.5 |
| Particle size after 0 min. | 126 nm | 124 nm |
| (mean) | | |
| Particle size after 20 min. | 124 nm | 288 nm |
| (mean) | | |

Measurements of particle size distribution were performed 0 minutes and 20 minutes after dispersion procedure was stopped. The results of the experiments are summarized in table 8b above. This table clearly shows that the mean particle size of formulation dispersed in neutral pH increases with time but no change in mean particle size is observed when the formulation is dispersed in acid media.

### EXAMPLE 5

Release of Particles of Fragmented Reversed Micellar (L₂) Phase Containing Progesterone or Cyclosporin A from Formulations Solidified with PVP

The composition of the formulations are described in Tables 9a and 9b.

TABLE 9a

| Substance | Amount (wt %) |
|---|---|
| CsA | 5 |
| Glycerolmonooleate (GMO) | 20 |
| GDO | 25 |
| PVP (Plasdone K29/32) | 50 |

TABLE 9b

| Substance | Amount (wt %) |
|---|---|
| Progesterone | 1.5 |
| GMO | 24.2 |
| GDO | 24.2 |
| PVP (Plasdone K29/32) | 50 |

On a weight basis, the formulations contain 50% wt of the PVP polymer with low molecular weight (Plasdone K29/32). The dry formulation was prepared in the following way: 1 wt % of the formulation in Table 8, excluding the PVP, was dispersed in water with the aid of sonication. To this solution the appropriate amount of PVP polymer was added before thorough mixing and lyophilization. After redissolution, lipid vehicles containing the drug substance formed and had an internal structure. These were particles of a fragmented reversed micellar (L₂) phase. The size distribution of the particles was not affected by the polymer or by the fact that they were released from a tablet.

### EXAMPLE 6

Release of Fragmented Lamellar Phase (Liposomes) Containing Cyclosporin A from Formulations Solidified with Low and High Molecular Weight PVP

This example illustrates that a molecularly mixed dry formulation, that on contact with an aqueous phase releases a fragmented lamellar phase (liposomes), can be obtained from mixing the components in a common solvent. After mixing the components the common solvent (EtOH) was removed by lyophilisation. The dry formulation could be compressed to a tablet

The composition of the formulations before and after lyophilization is given in Tables 10 and 11.

TABLE 10

| | Composition before | Composition after |
|---|---|---|
| Substance | lyophilization (%) | lyophilization (%) |
| CsA | 0.99 | 2.7 |
| Diglycerol monocaprinate | 8.02 | 22.3 |
| (DGMC) | | |
| Polyvinyl pyrrolidone | 27.0 | 75.0 |
| (PVP) (Plasdone K29/32) | | |
| Ethanol (EtOH) | 64.0 | — |

TABLE 11

| | Composition before | Composition after |
|---|---|---|
| Substance | lyophilization (%) | lyophilization (%) |
| CsA | 0.35 | 2.7 |
| Diglycerol | 2.86 | 22.3 |
| monocaprinate (DGMC) | | |
| Polyvinyl | 9.65 | 75.0 |
| pyrrolidone (PVP) | | |
| (Plasdone K90) | | |
| Ethanol (EtOH) | 87.1 | — |

Upon contacting the dry formulation with an aqueous phase (simulated intestinal fluid (SIF)) in excess, drug carrying particles of a fragmented lamellar phase (liposomes) formed. The size distribution of the liposomes was not affected by the polymer or by the fact that they were released from a dry and compressed tablet. Neither were the anisotropic properties of the liposomes changed (as investigated with a microscope equipped with crossed polarisers)

As in the earlier Examples, the release kinetics could be controlled by changing the solidifying polymer. The higher the molecular weight used, the slower the observed release.

### EXAMPLE 7

Release of Fragments of a Lamellar Phase (Liposomes) from a Formulation Solidified with PVP, and Control of the Size Distribution of the Released Liposomes

Liposomes can be obtained with reduced particle sizes by sonication before lyophilization. The reduced particle size is conserved in the dry tablet form and reappears on redissolution. Three size distributions were obtained in the following way. One formulation was obtained by emulgating the composition given in Table 12.

TABLE 12

| Substance | Amount (wt %) |
|---|---|
| CsA | 10 |
| Diglycerol monocaprinate (DGMC) | 81 |
| EtOH | 9 |

A second by first emulgating the composition of Table 12 in an aqueous solution that contained an appropriate amount of the PVP polymer, followed by lyophilization. The resulting composition had the constituents set out in Table 13.

TABLE 13

| | Composition after |
|---|---|
| Substance | lyophilization (%) |
| CsA | 2.7 |
| DGMC | 22.3 |
| Polyvinyl pyrrolidone | 75.0 |
| (PVP) (Plasdone K29/32) | |

A third composition, corresponding to the second in contents, was subject to sonication before the lyophilization. Sonication served to reduce the main mode released liposomal particle diameter from about 4 μm to about 0.4 μm.

### EXAMPLE 8

Effect of Decreased Size of the Liposomal Structures

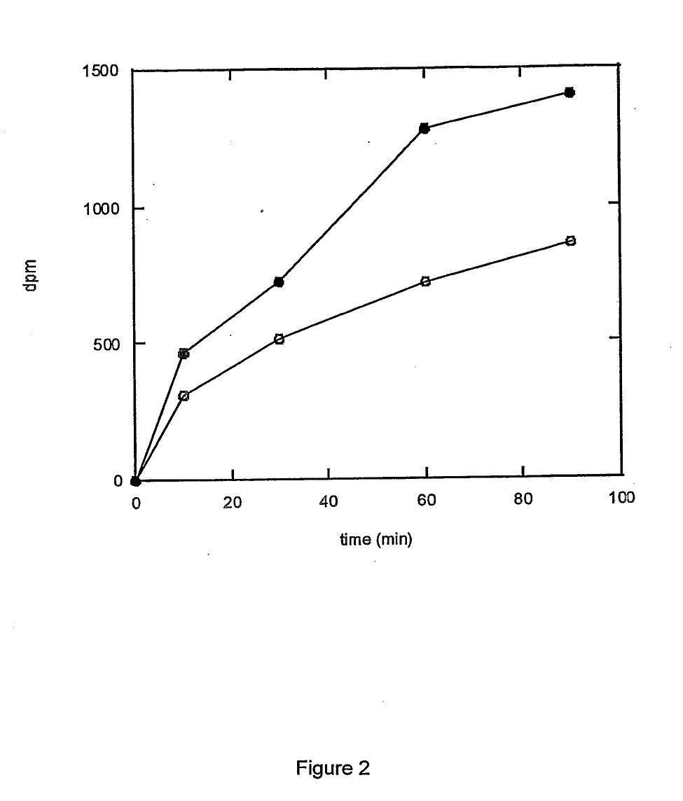

In Table 14 is given the composition of a formulation that on lyophilization gives fragmented lamellar (liposomal) structures. The size distribution of the liposomes can be decreased by sonication. Without sonification mode particle sizes were in the range 0.1 to 2 μm. With one minute sonication this was reduced to a single mode at about 0.1 μm. The formulation with the lower size distribution gives a higher uptake in vitro in the Ussing model as well as in vivo in the rat model using in situ perfusion (illustrated in FIG. 2 of the accompanying drawings). In FIG. 2, the time dependent ³H—CsA absorption into the intestinal cell membrane in the rat model using in situ perfusion of the proximal small intestine is plotted as a function of dpm (disintegrations per minute) against time for the composition of Table 14 without sonification (o) and with one minute sonication (•)

TABLE 14

| Substance | Amount (wt %) |
|---|---|
| CsA | 10 |
| Diglycerolmonooleate (DGMO) | 42 |
| Polysorbate 80 | 38 |
| EtOH | 10 |

### EXAMPLE 9

Release of Bilayer Structures Containing Bioactive Agent Amphotericin B from a Formulation Solidified with High Molecular Weight PVP

An Amphotericin B containing formulation was prepared in the following way;

(A) lyso-oleoyl phosphatidylcholine (LOPC, 24%), phosphatidylcholine (PC, 38%), cholesterol (5.2%), and ethanol (33%) were mixed over night to obtain a solution.

(B) High molecular weight polyvinyl pyrroloidone, Plasdone K-90, ISP Technologies, Inc, PVP (10%) and water (90%) were mixed over night to obtain a solution.

(C) Amphotericin B (AmB, 1.5%) was dissolved in a mixture (90/10) of glacial acetic acid/water. After 30 minutes the mixture was a homogeneous solution. The three solutions (A, B, and C) were mixed in the proportions 13/74/13 and after thorough mixing to a homogeneous solution the mixture was freeze dried to obtain a dry formulation, Table 15.

300 mg of the dry formulation was compressed to a tablet, which was placed in a basket (rotating at 100 rpm) and released in 500 ml simulated intestinal fluid (SIF). The release of AmB in fragmented bilayer carriers was monitored with UV-vis detection operating at 415 nm and 500 nm. The released liposomal drug carriers had a mean mode size below 1 μm.

TABLE 15

| | Composition before | Composition after |
|---|---|---|
| Substance | lyophilization (%) | lyophilization (%) |
| lyso-oleoyl | 3.12 | 19.10 |
| phosphatidylcholine | | |
| phosphatidylcholine | 4.94 | 30.25 |
| Cholesterol | 0.68 | 4.14 |
| EtOH | 4.29 | — |
| polyvinyl pyrroloidone | 7.40 | 45.31 |
| Plasdone K-90 | | |
| Amphotericin B, AmB | 0.195 | 1.19 |
| glacial acetic acid | 11.52 | — |
| Water | 67.88 | — |

### EXAMPLE 10

Release of Cubic Phase Particles Containing Cyclosporin A, Stabilised with Sucrose Ester of a Fatty Acid, from a Tablet Solidified with Polyethylene Glycol.

This example illustrates that cubic phase particles can form from a dry formulation in a self-dispersing process on contact with an aqueous phase.

A molecular mixture was obtained by mixing the components in Table 16. Two versions of the formulation were prepared differing only by the fatty acid sucrose ester. The sucrose esters that were used were either sucrose monopalmitate (Ryoto P-1570, Mitsubishi Kagaku) or sucrose monooleate (Ryoto O-1570, Mitsubishi Kagaku).

EtOH was evaporated from the molecular mixture to obtain a dry powder that could be compressed to a tablet. After contacting either of the two formulation with SIF (0.1%) a dispersion of cubic phase particles was formed. The particles had a broad size distribution (<100 μm).

TABLE 16

| | Composition before | Composition after |
|---|---|---|
| Substance | evaporation (%) | evaporation (%) |
| cyclosporine A, CsA | 2.5 | 10 |
| fatty acid sucrose ester | 2.5 | 10 |
| (sucrose monopalmitate or | | |
| sucrose monooleate) | | |
| glycerol monooleate, GMO | 7.5 | 30 |
| PEG (M_{w} = 4000) | 12.5 | 50 |
| EtOH | 75 | — |

### EXAMPLE 11

Release of Cubic Phase Particles, Containing a Bioactive Agent (Ketoconazole) with Weak Base Properties and Only Sparingly Soluble in SIF, from a Formulation Solidified with PEG.

(A) 0.5 g ketoconazole (KC) was dissolved in 50 g glacial acetic acid (HAc).

(B) 4.5 g of a solution consisting of 50% polyethylene glycol (PEG, M_{w}=35 000); 6% polysorbate 80 (P80); 6% Lutrol F127; 2% oleic acid (OA); and 36% glycerol monooleate (GMO), was molecularly mixed in the melted state at 70° C.

The (A) and (B) solutions were mixed at 70° C. to a homogeneous solution. Glacial acetic acid was removed by evaporation at 70° C. By lowering the temperature a solid formulation was formed, Table 17. The solid formulation was filled in capsules; 0.650 g in each capsule.

Release profiles were obtained by contacting 2 capsules with 500 ml simulated intestinal fluid (SIF) at 37° C. in an Erweka dissolution bath (a basket rotating at 100 rpm was used). Aliquots, 1 ml, were withdrawn at each time point and, after molecularly dissolving the cubic phase particles having contained therein KC by subsequently adding 0.75 ml 1.25M HCl in EtOH and 1.75 ml EtOH, the concentration of ketoconazole was assessed by UV-VIS absorbance at 276 nm.

The release of cubic phase particles having ketoconazole contained therein appeared 1st order and was finished after approximately 1.5 hours.

TABLE 17

| | Composition before | Composition after |
|---|---|---|
| Substance | evaporation (%) | evaporation (%) |
| ketoconazole, KC | 0.91 | 10.01 |
| glacial acetic acid, HAc | 90.91 | — |
| PEG (M_{w} = 35 000) | 4.09 | 44.99 |
| polysorbate 80, P80 | 0.49 | 5.39 |
| Lutrol F127 | 0.49 | 5.39 |
| oleic acid, OA | 0.16 | 1.76 |
| glycerol monooleate, GMO | 2.95 | 32.45 |

### EXAMPLE 12

Cubic Phase Particles Coated with Chitosan to Obtain a Positive Surface Charge.

A melt mixture of 90% glycerol monooleate (GMO) and 10% Lutrol F127 was dispersed in an aqueous solution with a homogeniser to a course dispersion (5% lipid by weight). By using a high pressure homogenisation (5 cycles at 5000 psi) the course dispersion was transformed to a finer dispersion (mean mode size 200 nm after autoclavation for 20 min at 120° C.). 20 ml of an aqueous solution (5% acetic acid) containing 0.5% chitosan was mixed with 20 ml of the 5% cubic phase lipid dispersion. This dispersion was found to consist of cubic liquid crystalline phase particles characterised by a mean mode size of 250 nm and a ζ-potential of +4 mV in the pH range pH=4 to 6.5. Above pH 7 the particles became uncharged.

### EXAMPLE 13

Release of Chitosan Coated Cubic Phase Particles from a Tablet Solidified with PEG.

A melt mixture of 90% glycerol monooleate (GMO) and 10% Lutrol F127 was dispersed in an aqueous solution with a homogeniser to a course dispersion (5% lipid by weight). By using high pressure homogenisation (5 cycles at 5000 psi) the course dispersion was transformed to a finer dispersion (mean mode size 400 nm after autoclavation for 20 min at 120° C.). 20 ml of a solution containing 0.5% chitosan (dissolved in an 5% acetic acid solution) was mixed with 20 ml of the 5% dispersion of cubic phase particles and 10 ml 10% PEG (M_{w}=4000) solution. pH was adjusted to pH=7.12 by titration with 1M NaOH. This dispersion was freeze dried to obtain a dry powder which could be compressed to a tablet, Table 18. After dissolution in 500 ml simulated intestinal fluid (SIF), cubic liquid crystalline phase particles with a broad size distribution (<100 μm) were obtained.

TABLE 18

| | Composition before | Composition after |
|---|---|---|
| Substance | lyophilization (%) | lyophilization (%) |
| glycerol | 1.8 | 42.86 |
| monooleate, GMO | | |
| Lutrol F127 | 0.2 | 4.76 |
| Water | 93.8 | — |
| chitosan | 0.2 | 4.76 |
| HAc | 2 | — |
| PEG (M_{w} = 4000) | 2 | 47.62 |

### EXAMPLE 14

Release of Chitosan Coated Cubic Phase Particles from a Tablet Solidified with Polyethylene Glycol.

4.5 g of a solution consisting of 50% polyethylene glycol (M_{w}=35 000), 6% polysorbate 80, 6% Lutrol F127, 2% oleic acid, and 36% glycerol monooleate, was molecularly mixed in the melted state at 70° C. By lowering the temperature a solid formulation was formed. The solid formulation was dispersed in water (5% solid components) to form cubic phase particles. 20 ml of a solution containing 0.5% chitosan (dissolved in an 5% acetic acid solution) was mixed with 20 ml of the 5% dispersion of cubic phase particles. pH was adjusted to pH=7.12 by titration with 1M NaOH. This dispersion was freeze dried to obtain a dry powder, Table 19. After dissolution in 500 ml simulated intestinal fluid, cubic liquid crystalline phase particles with a broad size distribution (<100 μm) were obtained.

TABLE 19

| | Composition before | Composition after |
|---|---|---|
| Substance | lyophilization (%) | lyophilization (%) |
| Polysorbate 80 | 0.15 | 5.45 |
| Glycerol | 0.90 | 32.72 |
| monooleate | | |
| Lutrol F127 | 0.15 | 5.45 |
| Water | 94.75 | — |
| chitosan | 0.25 | 9.09 |
| Glacial acetic | 2.50 | — |
| acid | | |
| Polyethylene | 1.25 | 45.45 |
| glycol | | |
| (M_{w} = 35 000) | | |
| Oleic acid | 0.05 | 1.82 |

1. An orally administrable dry composition comprising at least one physiologically tolerable polymer with dispersed therein particles comprising at least one physiologically tolerable lipid and a bioactive agent, which particles on contact with water or GI tract liquid form cubic phase, hexagonal phase or L₃ phase nanometre-sized particles containing said lipid, said bioactive agent and water and wherein said lipid comprises a diglyceride.

2. An orally administrable dry composition comprising a dry mixture of at least one physiologically tolerable polymer, at least one physiologically tolerable lipid and at least one bioactive agent, said lipid, bioactive agent and polymer being interdispersed at a molecular level and being capable on contact with water or GI tract liquid of forming cubic phase, hexagonal phase or L₃ phase particles comprising said lipid and said bioactive agent and optionally also water.

3. An orally administrable dry composition comprising at least one physiologically tolerable polymer with dispersed therein particles comprising at least one physiologically tolerable lipid and a bioactive agent, which particles on contact with water or GI tract liquid form nanometre-sized reversed hexagonal liquid crystalline particles containing said lipid, said bioactive agent and water.

4. A composition as claimed in claim 1 wherein said polymer is a hydrophilic water soluble polymer.

5. A composition as claimed in claim 1 wherein said polymer is a hydrophilic polymer capable of forming a gel when dissolved in aqueous solvent.

6. A composition as claimed in claim 1 wherein said particles contain water and are of a phase selected from normal cubic phase, reversed cubic phase, normal hexagonal phase, reversed hexagonal phase and L₃ phase.

7. A composition as claimed in claim 1 wherein said particles have a maximum dimension of 10 nm to 100 □m.

8. A composition as claimed in claim 1 additionally comprising a surfactant.

9. A composition as claimed in claim 8 wherein said surfactant is a sugar surfactant.

10. A composition as claimed in claim 1 herein said particles are surface modified with a surface active polymer.

11. A composition as calimed in claim 10 wherein said surface active polymer is selected from chitosan, chitosan derivatives, alginante, alginante derivatives, cellulose, cellulose derivatives and mixtures thereof.

12. A composition as in claim 1 wherein said particles are muco-adhesive.

13. A composition as claimed in claim 1 wherein said lipid comprises a swelling polar lipid selected from gatactolipids, lecithins, phosphatidylethanolamines, phosphatidylinositols, phosphatidylserines, sphingomyelins, monoglycerides, acidic soaps, cerebrosides, phosphatidic acids, plasmalogens, cardiolipins, di-glycerolesters of fatty acids, oligo-glycerolesters of fatty acids, poly-glycerolesters of fatty acids, di-glycerolethers of fatty alcohols, oligo-glycerolethers of fatty alcohols poly-glycerolethers of fatty alcohols and mixtures thereof.

14. A composition as claimed in claim 1 wherein said lipid comprises at least one fatty acid.

15. A composition as claimed in claim 14 wherein said composition releases particles in auqeous solution at pH below 3 and larger particles in auqeous solution at pH above 6, wherein the particles released at pH below 3 are sub-nanometer in size.

16. A composition as claimed in claim 1 wherein said polymer is selected from polysaccharides, cellulose derivatives, cellulose ethere, and synthetic polymers.

17. A composition as claimed in claim 16 wherein said polymer is a polysaccharide selected from starch, maltodextrin, carrageenan, xanthan gum, locus bean gum, acacia gum, chitosan, alginates, hyaluronic acid and pectin.

18. A composition as claimed in claim 1 wherein said composition forms 0.5 to 1000 nm particles upon exposure to aqueous fluids at pH below 7 and 250 to 10 000 nm particles at pH above 6.

19. A process for the production of an orally administrable composition, which process comprises removing solvent from a solution of at least one physiologically tolerable polymer, at least one physiologically tolerable lipid and at least one bioactive agent, and optionally grinding, compacting, coating and/or encapsulating the resultant solid.

20. A process for the production of an orally administrable composition, which process comprises melting and mixing a mixture of at least one physiologically tolerable hydrophilic polymer, at least one physiologically tolerable lipid and at least one bioactive agent, and optionally grinding, compacting, coating and/or encapsulating the resultant solid.

21. A process as claimed in claim 20 wherein said melting and mixing comprises melt extrusion.

22. A process for the production of a composition as claimed in claim 1, which process comprises removing solvent from a solution containing a dissolved physiologically tolerable water-soluble hydrophilic polymer and a dispersed physiologically tolerable lipid having dissolved or dispersed therein a bioactive agent.

23. A process for the production of an orally administrable composition which process comprises removing solvent from a solution containing a dissolved physiologically tolerable water-soluble hydrophilic polymer, a dissolved or dispersed bioactive agent and a dispersed physiologically tolerable lipid wherein the lipid is dispersed in the solution in the form of structured particles.

24. A process as claimed in claim 19 wherein the removal of said solvent is carried out by lyophilisation or spray drying.

25. A pharmaceutical formulation comprising a composition as claimed in claim 1 and optionally at least one pharmaceutically acceptable carrier or excipient.

26. A pharmaceutical formulation as claimed in claim 25 comprising a composition pressed into the form of a tablet.

27. A pharmaceutical formulation as claimed in claim 25 comprising progesterone.
